(19) United States
(12) Patent Application Publication
(10) Pub. No.: US 2021/0102900 A1
(43) Pub. Date: Apr. 8, 2021

(54) BIOSENSOR BASED ON TRITITANIUM DICARBIDE TWO-DIMENSIONAL METAL CARBIDE CATALYZED LUMINOL ELECTROGENERATED CHEMILUMINESCENCE PROBE AND PREPARATION METHOD

(71) Applicant: QINGDAO UNIVERSITY, Qingdao, Shandong (CN)

(72) Inventors: Zonghua WANG, Qingdao (CN); Huixin ZHANG, Qingdao (CN); Yang LIU, Qingdao (CN)

(73) Assignee: QINGDAO UNIVERSITY, Qingdao, Shandong (CN)

(21) Appl. No.: 16/607,911

(22) PCT Filed: Nov. 26, 2018
(86) PCT No.: PCT/CN2018/117512

§ 371 Date: Oct. 24, 2019

(30) Foreign Application Priority Data
Apr. 20, 2018 (CN) 2018103580948

(51) Int. Cl. G01N 21/76; G01N 33/543; G01N 33/551; C09K 11/07; B01J 31/22; G01N 27/30; G01N 27/327

(52) U.S. Cl. CPC G01N 21/76; G01N 33/5438; G01N 33/551; G01N 2021/757; B01J 31/2295; G01N 27/308; G01N 27/3278; C09K 11/07

(57) ABSTRACT

An electrogenerated chemiluminescence (ECL) probe is based on trititanium dicarbide two-dimensional (2D) metal carbide catalyzed luminol and a preparation method. The biosensor includes the probe and the electrode of the biosensor, wherein the probe includes the Ti₃C₂ MXenes nanosheets, a linker molecule and a bio-recognition molecule 1; the Ti₃C₂ MXenes nanosheets are linked with the linker molecule by electrostatic adsorption; the linker molecule is linked with the bio-recognition molecule 1 by an amide group, contains a primary or secondary amine group, and presents positive potential in water; the bio-recognition molecule 1 is a single-stranded DNA sequence 1 having a carboxyl group at the 5′ end, and a CD63 protein on exosomes is recognized by the single-stranded DNA sequence 1. It was found for the first time that Ti₃C₂ MXenes can improve the ECL signal of luminol, the Ti₃C₂ MXenes could be applicable to the ECL probe.

### FIELD OF THE INVENTION

The present invention relates to the field of materials and analytical chemistry, in particular to a novel two-dimensional (2D) nanomaterial, i.e., Ti₃C₂ MXenes material which catalyzes electrogenerated chemiluminescence (ECL) signal of luminol, and a method for constructing an ECL biosensor by using the carboxyl-terminated poly(N-isopropylacrylamide) (carboxyl-terminated PNIPAM) polymer molecules to expose more active sites at a suitable temperature so as to detect exosomes.

### BACKGROUND OF THE INVENTION

Exosomes are nanoscale extracellular vesicles (30˜100 nm) released from multivesicular bodies by an endolysosomal pathway. Exosomes carry abundant cellular genetic materials, including transmembrane proteins and cytoplasmic proteins, mRNA, DNA, and microRNA, and thereby act as mediators between mediate cells. They play an important role. And experiments have shown that they are related to diseases, especially related to the pathogenesis of cancer. Exosomes are considered as biomarker for early cancer diagnosis, and have important significance in cancer diagnosis. So far, various methods for exosomes detection have been developed, including western blot, flow cytometry, or enzyme-linked immunosorbent. These methods have the disadvantages of requiring expensive instruments, complex technical skills and time-consuming operations, etc. Therefore, it is a huge challenge to develop a simple, sensitive and reliable detection method of exosomes. In recent years, ECL as a powerful analytical technique has been widely used for the detection of some substances such as proteins, DNA and enzymes, owing to its high sensitivity, rapidness, low background noise, easy controllability, low cost and the like. Therefore, its numerous advantages make it promising in the detection of exosomes. MXenes are novel 2D early transition metal family carbides. MXenes are prepared by selectively etching an Al element from metal-conducting MAX phases, wherein the MAX phases include various types such as Ti₂AlC, Ti₃AlC₂ and Ti₄AlC₃. Ti₃C₂ MXenes are one of them, combining the metal conductivity of transition metal carbides with the hydrophilic property of a hydroxyl or oxygen terminated surface. In essence, they behave as “conductive clay”. They have some properties such as conductivity, catalysis and large specific surface area, which are similar to those of graphene. Therefore, based on these excellent properties, Ti₃C₂ MXenes show great prospects in numerous applications, such as catalysis, biosensors, contaminant treatment, supercapacitors and lithium ion batteries. However, up to now, there have been few reports on the application of Ti₃C₂ MXenes in biosensors and biomedicine such as cancer treatment, cell uptake and antibacterial activity. Therefore, based on the excellent catalytic properties and conductivity, Ti₃C₂ MXenes have shown the potential to produce highly sensitive ECL biosensors.

### SUMMARY OF THE INVENTION

In order to overcome the deficiencies of the prior art, one of the objectives of the present invention is to provide a probe of an ECL biosensor, which can improve the ECL signal of luminol.

In order to achieve the above objective, the technical solution of the present invention is:

An ECL probe based on trititanium dicarbide 2D metal carbide catalyzed luminol, including Ti₃C₂ MXenes nanosheets, a linker molecule and a bio-recognition molecule 1, wherein the Ti₃C₂ MXenes nanosheets are linked with the linker molecule by electrostatic adsorption; the linker molecule is linked with the bio-recognition molecule 1 by an amide group, contains a primary or secondary amine group and presents positive potential in water; the bio-recognition molecule 1 is the single-stranded DNA sequence 1 having a carboxyl group at the 5′ end, and the CD63 protein on exosomes is recognized by single-stranded DNA sequence 1.

The inventors of the present invention have found for the first time that the Ti₃C₂ MXenes can improve the ECL signal of luminol, so the Ti₃C₂MXenes could be applicable to the preparation of ECL probe. After further research, it was found that the Ti₃C₂ MXenes nanosheets present negative potential in water. Therefore, the substance having a positive charge and an amino group in water was used to link with the Ti₃C₂ MXenes nanosheets, which facilitates the linking of the Ti₃C₂ MXenes with the single-stranded DNA sequence 1. Thereby an ECL probe of 2D metal carbide as a carrier is obtained.

A second objective of the present invention is to provide a preparation method of the probe, the method including: of mixing linker molecules with Ti₃C₂ MXenes nanosheets uniformly in water, stirring for a period of time and centrifuging to obtain sediment, and performing an amide reaction on the obtained sediment and bio-recognition molecule 1 to obtain the probe.

A third objective of the present invention is to provide the electrode of the biosensor, for use with the probe, wherein a surface of a glassy carbon electrode (GCE) is modified by gold nanoparticles (AuNPs). The AuNPs are linked with one amino group in a molecule containing at least two amino groups by an amide group, an other amino group in the molecule is linked with one carboxyl group in the carboxyl-terminated PNIPAM by an amide group such that the carboxyl-terminated PNIPAM is linked with the molecule, an other carboxyl group of the carboxyl-terminated PNIPAM is linked with the bio-recognition molecule 2 by an amide group such that the carboxyl-terminated PNIPAM is linked with the bio-recognition molecule 2. The bio-recognition molecule 2 is a single-stranded DNA sequence 2 carrying an amino group at the 5′ end, and the single-stranded DNA sequence 2 is capable of recognizing the EpCAM protein on exosomes.

The surface of the AuNPs contain a carboxyl group, which are linked with the carboxyl-terminated PNIPAM by the molecule containing at least two amino groups. The polymer chain of the carboxyl-terminated PNIPAM is extended to expose active sites of multiple aptamers at room temperature, so that more exosomes are captured in the electrode.

A fourth objective of the present invention is to provide a method for preparing the electrode of the biosensor, including the steps of dropping AuNPs solution onto the surface of the GCE such that AuNPs are attached to the surface of the GCE, linking molecules containing at least two amino groups to the AuNPs by the amide reaction, then linking carboxyl-terminated PNIPAM with the molecules containing at least two amino groups by the amide reaction, and then linking the bio-recognition molecules 2 with the carboxyl-terminated PNIPAM by the amide reaction.

A fifth objective of the present invention is to provide an ECL biosensor including the probe and the electrode of the biosensor.

A sixth objective of the present invention is to provide an ECL kit including the probe, the electrode of the biosensor and luminol.

A seventh objective of the present invention is to provide an application of the probe, the electrode of the biosensor, the biosensor or the kit in the ECL biosensor detection of exosomes.

An eighth objective of the present invention is to provide a method for detecting exosomes by an ECL biosensor, including the steps of immersing the electrode of the biosensor into the exosomes solution to be detected such that the exosomes are attached to the electrode of the biosensor, then immersing the electrode of the biosensor carrying the exosomes into the solution of the probe such that the probes are attached to the exosomes on the electrode of the biosensor to constitute a biosensor consisting of the probes and the electrode of the biosensor loading the exosomes, and the modified electrode was used for subsequent ECL characterization.

Advantageous effects of the present invention are:

It is found for the first time in the present invention that Ti₃C₂ MXenes can improve the ECL signals of luminol, and using this property to prepare Ti₃C₂ MXenes as probes, then the electrode of the biosensor was obtained and the biosensor was thus obtained. The ECL signal intensities were linear with the logarithm of the exosomes concentration in the range from 5.0×10⁵-5×10⁹ particles/mL. The detection limit was 2.5×10⁵ particles/mL with a correlation coefficient of R=0.9740.

### BRIEF DESCRIPTION OF THE DRAWINGS

The accompanying drawings constituting a part of the present application are used for providing a further understanding of the present application, and the illustrative embodiments of the present application and the description thereof are used for interpreting the present application, rather than constituting improper limitations to the present application.

FIG. 1 is a preparation mechanism diagram of an ECL biosensor;

FIG. 2 is a scanning electron microscopy (SEM) photograph of Ti₃C₂ MXenes prepared in embodiment 1;

FIG. 3 is a diagram showing the relationship between the ECL intensity of the ECL biosensor prepared in embodiment 1 and the concentration of exosomes, where a is 5.0×10⁵ particles/mL, and j is 5.0×10⁹ particles/mL.

### DETAILED DESCRIPTION OF THE EMBODIMENTS

It should be pointed out that the following detailed descriptions are all exemplary and aim to further illustrate the present application. Unless otherwise specified, all technological and scientific terms used in the descriptions have the same meanings generally understood by those of ordinary skill in the art of the present application.

It should be noted that the terms used herein are merely for describing specific embodiments, but are not intended to limit exemplary embodiments according to the present application. As used herein, unless otherwise explicitly stated by the context, the singular form is also intended to include the plural form. In addition, it should also be appreciated that when the terms “include” and/or “comprise” are used in the description, they indicate features, steps, operations, devices, components and/or their combination.

Luminol mentioned in the present invention is also referred to as luminescent ammonia. Its chemical name is 3-aminophthalhydrazide. It is a blue crystal or beige powder at room temperature, and is a relatively stable synthetic organic compound. Its chemical formula is C₈H₇N₃O₂.

The amide reaction described in the present application refers to a process of reacting a carboxyl group with a primary or secondary amine group to generate an amide group.

As described in the background, there are few records in the prior art about the application of Ti₃C₂ MXenes in biosensors and biomedicine such as cancer treatment, cell uptake and antibacterial activity. In order to solve the technical problems, the present application provides a biosensor of the ECL probe based on trititanium dicarbide 2D metal carbide catalyzed luminol and a preparation method.

A typical embodiment of the present application provides an ECL probe based on trititanium dicarbide 2D metal carbide catalyzed luminol, including Ti₃C₂ MXenes nanosheets, a linker molecule and a bio-recognition molecule 1. The Ti₃C₂ MXenes nanosheets are linked with the linker molecule by electrostatic adsorption. The linker molecule is linked with the bio-recognition molecule 1 by an amide group, contains a primary or secondary amine group and presents positive potential in water. The bio-recognition molecule 1 is the single-stranded DNA sequence 1 having the carboxyl group at the 5′ end and the single-stranded DNA sequence 1 is capable of recognizing the CD63 protein on exosomes.

The inventors of the present invention have found for the first time that Ti₃C₂ MXenes can improve the ECL signal of luminol, so the Ti₃C₂ MXenes were desired to be prepared into the probe of an ECL biosensor. However, it was difficult to modify the Ti₃C₂ MXenes in the modification process. After further research, it was found that the Ti₃C₂ MXenes nanosheets present negative potential on its surface in water. Thus, a linker molecule presents positive potential was used to link the Ti₃C₂ MXenes nanosheets with the single-stranded DNA sequence 1, thus obtaining the ECL probe.

Preferably, the linker molecule is polyethylene imine (PEI), having a weight average molecular weight of 70,000. Polyethylene imine is a water-soluble polymer compound. When the polyethylene imine is dissolved in water, a large amount of positive charge is distributed on the surface of the polyethylene imine in the aqueous solution, and can electrostatically adsorb the Ti₃C₂ MXenes nanosheets.

Preferably, the sequence of the single-stranded DNA sequence 1 from 5′ to 3′ is TTTTTT CAC CCC CAC CTC GCT CCC GTG ACA CTA ATG CTA (SEQ ID NO. 1).

The present application provides a preparation method of the probe, including the steps of mixing linker molecules with Ti₃C₂ MXenes nanosheets uniformly in water, stirring for a period of time and centrifuging to obtain these diment, and performing the amide reaction on the obtained sediment and bio-recognition molecule 1 to obtain the probe.

Preferably, the stirring time is 1 to 1.5 h. The revolution speed of centrifugation is more than 10,000 rpm.

Preferably, the reaction system of the amide reaction includes 1-(3-(dimethyl-amino)propyl)-3-ethylcarbodiimidehydrochloride (EDC) and N-hydroxysuccinimide sodium salt (NHS).

The present application preferably discloses a method for etching Ti₃AlC₂, including the steps of immersing Ti₃AlC₂ in 48±2% (by mass) HF and stirring for 24±0.5 h at 45±2° C., centrifuging the powder particles at 4500 to 5500 rpm, washing 5 to 6 times for 5 min each time, discarding the supernatant, and drying at room temperature to obtain multilayer Ti₃C₂Tₓ particles.

The present application preferably discloses a method for preparing Ti₃C₂ MXenes nanosheets, including the steps of immersing the multilayer Ti₃C₂Tx particles in 1 mL DMSO and stirring for 24±0.5 h at room temperature. Then adding deionized water (DI water), smashing in a cell lysis instrument, and then centrifuging to obtain a colloidal solution of Ti₃C₂ MXenes. Further preferably, the centrifugation revolution speed before smashing is more than 10,000 rpm, more preferably 12,000 rpm, and the revolution speed after smashing is 3,000 to 4,000 rpm, more preferably 3,500 rpm.

The present invention provides the electrode of the biosensor for use with the probe, wherein the surface of the GCE is modified by AuNPs. The AuNPs are linked with one amino group in a molecule containing at least two amino groups by an amide group, the other amino group in the molecule is linked with one carboxyl group in the carboxyl-terminated PNIPAM by an amide group such that the carboxyl-terminated PNIPAM is linked with the molecule, the other carboxyl group of the carboxyl-terminated PNIPAM is linked with the bio-recognition molecule 2 by an amide group such that the carboxyl-terminated PNIPAM is linked with the bio-recognition molecule 2. The bio-recognition molecule 2 is a single-stranded DNA sequence 2 carrying an amino group at the 5′ end, and the single-stranded DNA sequence 2 is capable of recognizing the EpCAM protein on exosomes.

The surface of the AuNPs contain the carboxyl group, which are linked with the carboxyl-terminated PNIPAM by the linker molecule, and the polymer chain of the carboxyl-terminated PNIPAM is extended to expose active sites of multiple aptamers at room temperature, so that more exosomes could be captured in the electrode.

The molecule containing at least two amino groups may be ethylenediamine, propylene diamine, p-phenylenediamine, diaminooctane, propylene triamine, or diethylene tetramine. Preferably, the molecule containing at least two amino groups in the present application is ethylenediamine.

Preferably, the carboxyl-terminated PNPAM has a number average molecular weight of 1,000 to 5,000, from SIGMA-ALORICH.

Preferably, the sequence of the single-stranded DNA sequence 2 from 5′ to 3′ is TTTTTT CAC TAC AGA GGT TGC GTC TGT CCC ACG TTG TCA TGG GGG GTT GGC CTG (SEQ ID NO. 2).

The present application provides a preparation method of the electrode of the biosensor, including the steps of dropping AuNPs solution onto the surface of GCE such that AuNPs are attached to the surface of the GCE, linking molecules containing at least two amino groups to the AuNPs by the amide reaction, then linking carboxyl-terminated PNIPAM with the molecules containing at least two amino groups by the amide reaction, and then linking bio-recognition molecules 2 with the carboxyl-terminated PNIPAM by the amide reaction.

Preferably, the reaction temperature and the treatment temperature involved in the preparation method are 37±0.5° C., e.g., the amide reaction temperature, the treatment temperature at which the AuNPs are attached to the surface of the GCE, etc.

The GCE needs to be pretreated to clean the surface before the AuNPs are attached thereto. Preferably, the pretreatment of the GCE includes first polishing and then washing.

The present application also provides an ECL biosensor, including the probe and the electrode of the biosensor.

The present application also provides an ECL kit, including the probe, the electrode of the biosensor and luminol.

The present application also provides an application of the probe, the electrode of the biosensor, the ECL biosensor or the ECL kit in detecting exosomes by ECL.

The present application also provides a method for detecting exosomes by ECL, including the steps of immersing the electrode of the biosensor into the exosomes solution to be detected such that exosomes are attached to the electrode of the biosensor, then immersing the electrode of the biosensor carrying the exosomes into the solution of the probes such that probes are attached to the exosomes surface to constitute the biosensor consisting of the probes and the electrode of the biosensor carrying the exosomes, finally, the probe modified electrode was used for subsequent ECL characterization.

In order that those skilled in the art can understand the technical solutions of the present invention more clearly, the technical solutions of the present invention will be described in detail below with reference to specific embodiments.

### Materials

Aptamer1: 5′-COOH-TTTTTT CAC CCC CAC CTC GCT CCC GTG ACA CTA ATG CTA, and aptamer2: 5′-NH₂-TTTTTT CAC TAC AGA GGT TGC GTC TGT CCC ACG TTG TCA TGG GGG GTT GGC CTG were obtained from Shanghai Sangon Biological Engineering Technology & Services Co., Ltd. Ti₃AlC₂ (98%) was purchased from Forsman Scientific Co., Ltd. (Beijing, China). The carboxyl-terminated PNIPAM (PNIPAM, Mn=2000) and luminol were purchased from Sigma-Aldrich. HAuCl₄·3H₂O (48%, w/w) was obtained from Shanghai Reagent (Shanghai, China). 1-(3-(dimethyl-amino)propyl)-3-ethylcarbodiimidehydrochloride (EDC) and N-hydroxysuccinimide sodium salt (NETS), ethylenediamine (EDA) and dimethyl sulfoxide (DMSO) were all purchased from Beijing Chemical Co., Ltd (Beijing, China).

### Embodiment 1

### Synthesis of MXenes-Aptamer1 Nanoprobe

Ti₃AlC₂ (1.0 g) powder was immersed in 15 mL of 48% (by mass) HF, and was stirred for 24 h at 45° C. Then, the suspensions were centrifuged to separate solids from the supernatant. After that the solid products were washed several times at 5000 rpm for 5 minutes each time, and were dried at room temperature. The layered Ti₃C₂Tx was obtained and stored at 4° C. until use The demixed Ti₃C₂ (0.05 g) powder was immersed in 1 mL of DMSO, stirred at room temperature for 24 h, centrifuged at 12,000 rpm and washed five times for 5 min each time, then the supernatant was discarded and DI water was added for smashing in a cell lysis instrument for 2 h. Finally, the solution was centrifuged at 3500 rpm for 60 min, and the supernatant (i.e., Ti₃C₂ MXenes nanosheets dispersion) was retained and stored at 4° C. for later use. Its structural characteristic is shown in FIG. 2.

200 μL of (0.005 g/mL) PEI, 3 ml Ti₃C₂ MXenes nanosheets and 2 mL DI water were mixed, and were slowly stirred for 1 h at room temperature. Then, the suspensions were centrifuged at 12,000 rpm for 10 minutes to separate solids from the mixture. In addition, the aptamer1 (1 μM, 5′-COOH-TTTTTT CAC CCC CAC CTC CTC GCT CCC GTG ACA CTA ATG CTA) was activated by EDC (400 mM) and NHS (100 mM) for 1 h at 37° C. After that, 200 μL MXenes-PEI solution was added into the aptamer1 mixture solution (120 μL) for 1 h at 37° C. Finally, the mixture including EDC NHS, aptamer1 and MXenes-PEI was centrifuged at 12000 rpm for 10 minutes, the supernatant was discarded and DI water was added.

### Surface Pretreatment of GCE

The GCE was processed with 0.3 and 0.05 μM α-Al₂O₃ powder and rinsed ultrasonically with ethanol and DI water for 3 min, respectively, finally the surface of the electrode was dried with pure N₂.

The cleaned and blown GCE was used as a working electrode, Ag/AgCl was used as a reference electrode, and a platinum wire was used as a counter electrode. In a potassium ferricyanide solution, the GCE was scanned by voltammetry (CV) under −0.2 to 0.6 V at 100 mV/s till being stable. This operation was repeated till the redox potential difference of the GCE reached an activation standard of 80 mV, and then the GCE was washed with water and blown dry with N₂.

### Assembly of Electrode

GCE after AuNPs modification: Drop 6 μL of AuNPs solution on pretreated GCE (preparation method of AuNPs solution: 100 mL of 0.01% (w/v) HAuCl₄ solution was boiled with vigorous stirring, and then 0.588 mL of 0.2 mol/mL trisodium citrate solution was quickly added to the boiling solution. The solution turned dark red, indicating the formation of AuNPs. The solution was continuously stirred and cooled. The colloid was stored at 4° C. for later use, the electrode was incubated to be dry at 37° C., and then the electrode was immersed in 120 μL of mixture solution containing 2 mg mL⁻¹ EDA, 400 μM EDC and 100 μM NHS at 37° C. for 2 h. At the same time, 40 μL, 1 mg mL⁻¹ carboxyl-terminated PNIPAM was activated by 400 μM EDC and 100 μM NHS at 37° C. for 1 h. The GCE incubated in the EDA was further immersed in the carboxyl-terminated PNIPAM solution activated for 1 h and was incubated for 1 h. The immobilization of aptamer2 was finished by incubating the above electrode in 40 μL of 1 μM aptamer2 solution at 37° C. for 2 h washed and blown dry to obtain the electrode of the biosensor, which was recorded as aptamer2/PNIPAM/AuNPs/GCE.

### Assembly of Sensor

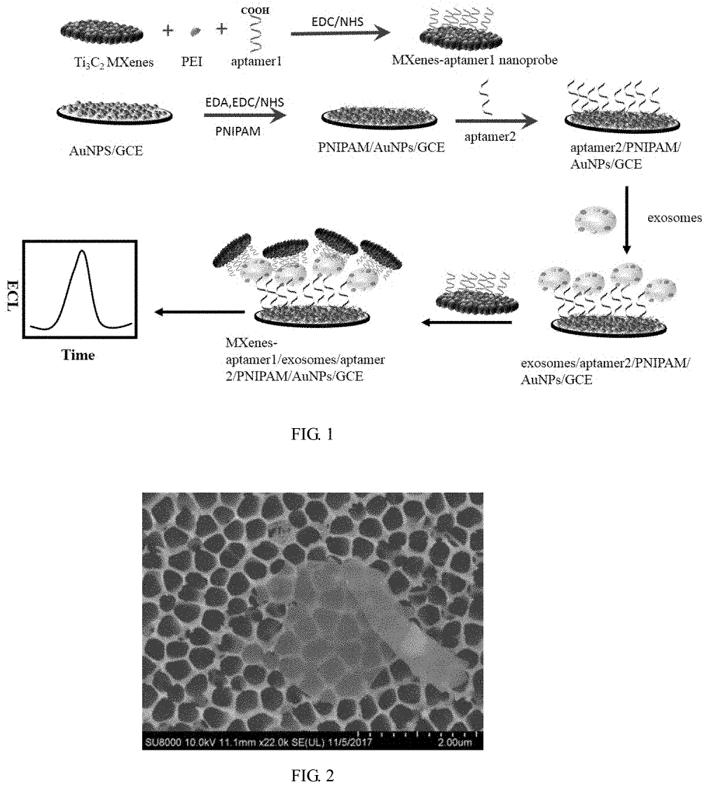

The aptamer2/PNIPAM/AuNPs/GCE was immersed in exosomes solution (5.0×10⁵-5×10⁹ particles/mL) at 37° C. for 2 h to form exosomes/aptamer2/PNIPAM/AuNPs/GCE. Finally the electrode which has captured exosomes was washed with DI water and blown dry. Then the above electrode was incubated in probe solution for 2 h at 37° C. After the reaction was completed, the electrode was washed with DI water, and blown dry with N₂ to obtain an ECL biosensor. The preparation process of the sensor is as shown in FIG. 1.

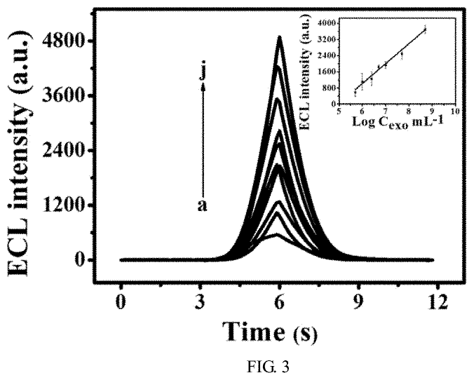

ECL detection was performed on the prepared sensor, and the detection results are shown in FIG. 3. The concentrations of the exosomes were 5.0×10⁵ particles/mL (a), 1×10⁶ particles/mL (b), 2.5×10⁶ particles/mL (c), 5×10⁶particles/mL (d), 10⁷ particles/mL (e), 5×10⁷particles/mL (f), 10⁸ particles/mL (g), 5×10⁸ particles/mL (h), 10⁹ particles/mL (i), 5×10⁹ particles/mL (j). It can be seen that the ECL signal of luminol improved with increasing concentrations of exosomes. And the ECL signal was linear with the logarithm of the exosomes concentration in the range from 5.0×10⁵-5×10⁹ particles/mL. The detection limit was 2.5×10⁵ particles/mL with a correlation coefficient of R=0.9740.

At the same time, the prepared ECL biosensor may also be used for detecting different exosomes such as MCF-7 (breast cancer cells), HepG2 (human liver cancer cell line) and B16 (melanoma cells) exosomes. The concentration of all three exosomes is 10⁷ particles/mL and the ECL signals produced were different. Among them, the ECL signal for detecting MCF-7 exosomes were the largest and the ECL signal for detecting B16 exosomes was the smallest. The fact shows that the designed ECL biosensor has excellent selectivity.

### Embodiment 2

This embodiment is the same as Embodiment 1, and the difference lies in that:

### Assembly of Electrode

GCE after AuNPs modification: Drop 6 μL of AuNPs (18 nm) solution on pretreated GCE, the electrode was incubated to be dry at 37° C., and then the electrode was immersed in 120 μL of mixture solution containing 2 mg mL⁻¹ EDA, 400 μM EDC and 100 μM NHS at 37° C. for 2 h. At the same time, 1 mg mL⁻¹ carboxyl-terminated PNIPAM was activated by 400 μM EDC and 100 μM NHS at 37° C. for 1 h. The GCE incubated in the EDA was further immersed in the PNIPAM solution activated for 1 h and was incubated for 1 h. The immobilization of aptamer2 was finished by incubating the above electrode in 40 μL of 0.8 μM aptamer2 solution at 37° C. for 2 h, washed and blown dry to obtain a the electrode of the biosensor, which was recorded as aptamer2/PNIPAM/AuNPs/GCE.

### Assembly of Sensor

The aptamer2/PNIPAM/AuNPs/GCE was immersed in exosomes with different concentrations at 25° C. for 1 h. And then, the exosomes captured electrode (exosomes/aptamer2/PNIPAM/AuNPs/GCE) was carefully rinsed with DI water.

The electrode which has captured exosomes was washed with distilled water and blown dry. The probe was modified in exosomes/aptamer2/PNIPAM/AuNPs/GCE at 37° C. for 1 h. After the reaction was completed, the electrode was washed with DI water, and blown dry with N₂ to obtain a prepared ECL biosensor.

### Embodiment 3

This embodiment is the same as Embodiment 1, and the difference lies in that:

### Assembly of Electrode

GCE after AuNPs modification: Drop 6 μL of AuNPs solution on pretreated GCE, the electrode was incubated to be dry at 37° C., and then the electrode was immersed in 120 μL of mixture solution containing 2 mg mL⁻¹ EDA, 400 μM EDC and 100 μM NHS at 37° C. for 2 h. At the same time, 1 mg mL⁻¹ carboxyl-terminated PNIPAM was activated by 400 μM EDC and 100 μM NHS at 37° C. for 1 h. The GCE incubated in the EDA was further immersed in the PNIPAM solution activated for 1 h and was incubated for 1 h. The immobilization of aptamer2 was finished by incubating the above electrode in 40 μL of 1.2 μM aptamer2 solution at 37° C. for 1.5 h, washed and blown dry to obtain the electrode of the biosensor, which was recorded as aptamer2/PNIPAM/AuNPs/GCE.

### Assembly of Sensor

The aptamer2/PNIPAM/AuNPs/GCE was immersed in exosomes with different concentrations at 50° C. for 30 min. And then, the exosomes captured electrode (exosomes/aptamer2/PNIPAM/AuNPs/GCE) was carefully rinsed with DI water.

The electrode which has captured exosomes was washed with DI water and blown dry. The probe was modified in exosomes/aptamer2/PNIPAM/AuNPs/GCE at 37° C. for 30 min. After the reaction was completed, the electrode was washed with DI water, and blown dry with N₂ to obtain a prepared ECL biosensor.

Described above are merely preferred embodiments of the present application, and the present application is not limited thereto. Various modifications and variations may be made to the present application for those skilled in the art. Any modification, equivalent substitution, improvement or the like made within the spirit and principle of the present application shall fall into the protection scope of the present application.

1. An electrogenerated chemiluminescence (ECL) probe based on trititanium dicarbide two-dimensional (2D) metal carbide catalyzed luminol, the ECL probe comprising Ti₃C₂ MXenes nanosheets, a linker molecule and a bio-recognition molecule 1, wherein the Ti₃C₂ MXenes nanosheets are linked with the linker molecule by electrostatic adsorption;
the linker molecule is linked with the bio-recognition molecule 1 by an amide group, contains a primary or secondary amine group and presents positive potential in water;
the bio-recognition molecule 1 is a single-stranded DNA sequence 1 having a carboxyl group at the 5′ end, and a CD63 protein on exosomes is recognized by the single-stranded DNA sequence 1;
the linker molecule is polyethylene imine.

2. The probe according to claim 1, wherein the sequence of the single-stranded DNA sequence 1 from 5′ to 3′ is TTTTTT CAC CCC CAC CTC GCT CCC GTG ACA CTA ATG CTA.

3. A method for preparing the probe according to claim 1, the method comprising steps of mixing the linker molecules with the Ti₃C₂ MXenes nanosheets uniformly in water, stirring for a period of time and centrifuging to obtain a sediment, and performing an amide reaction on the obtained sediment and the bio-recognition molecule 1 to obtain the probe;
the stirring time is 1 to 1.5 h; the revolution speed of centrifugal separation is more than 10,000 rpm;
preferably, a reaction system of the amide reaction comprises 1-(3-(dimethyl-amino)propyl)-3-ethylcarbodiimidehydrochloride and N-hydroxysuccinimide sodium salt.

4. An electrode of a biosensor for use with the probe according to claim 1, wherein a surface of a glassy carbon electrode (GCE) is modified by gold nanoparticles (AuNPs), the AuNPs are linked with one amino group in a molecule containing at least two amino groups by an amide group, an other amino group in the molecule is linked with one carboxyl group in a carboxyl-terminated poly(N-isopropylacrylamide) (carboxyl-terminated PNIPAM) by an amide group such that the carboxyl-terminated PNIPAM is linked with the molecule, an other carboxyl group of the carboxyl-terminated PNIPAM is linked with a bio-recognition molecule 2 by an amide group such that the carboxyl-terminated PNIPAM is linked with the bio-recognition molecule 2, wherein
the bio-recognition molecule 2 is a single-stranded DNA sequence 2 carrying an amino group at the 5′ end, and the single-stranded DNA sequence 2 is capable of recognizing an EpCAM protein on exosomes;
the carboxyl-terminated PNIPAM has a number average molecular weight of 1,000 to 5,000.

5. The electrode of the biosensor according to claim 4, wherein the sequence of the single-stranded DNA sequence 2 from 5′ to 3′ is TTTTTT CAC TAC AGA GGT TGC GTC TGT CCC ACG TTG TCA TGG GGG GTT GGC CTG.

6. A method for preparing the electrode of the biosensor according to claim 4, comprising the steps of dropping AuNPs solution onto the surface of the GCE such that AuNPs are attached to the surface of the GCE, linking the molecules containing at least two amino groups with the AuNPs by the amide reaction, then linking carboxyl-terminated PNIPAM with the molecules containing at least two amino groups by the amide reaction, and then linking the bio-recognition molecules 2 with the carboxyl-terminated PNIPAM by the amide reaction;
a reaction temperature and a treatment temperature involved in the method are 37±0.5° C.

7. An ECL biosensor, the biosensor comprising the probe according to claim 1, and the electrode of the biosensor, wherein a surface of a glassy carbon electrode (GCE) is modified by gold nanoparticles (AuNPs), the AuNPs are linked with one amino group in a molecule containing at least two amino groups by an amide group, an other amino group in the molecule is linked with one carboxyl group in a carboxyl-terminated poly(N-isopropylacrylamide) (carboxyl-terminated PNIPAM) by an amide group such that the carboxyl-terminated PNIPAM is linked with the molecule, an other carboxyl group of the carboxyl-terminated PNIPAM is linked with a bio-recognition molecule 2 by an amide group such that the carboxyl-terminated PNIPAM is linked with the bio-recognition molecule 2, wherein
the bio-recognition molecule 2 is a single-stranded DNA sequence 2 carrying an amino group at the 5′ end, and the single-stranded DNA sequence 2 is capable of recognizing an EpCAM protein on exosomes;
the carboxyl-terminated PNIPAM has a number average molecular weight of 1,000 to 5,000.

8. An ECL kit, the kit comprising the probe according to claim 1, and the electrode of the biosensor, wherein a surface of a glassy carbon electrode (GCE) is modified by gold nanoparticles (AuNPs), the AuNPs are linked with one amino group in a molecule containing at least two amino groups by an amide group, an other amino group in the molecule is linked with one carboxyl group in a carboxyl-terminated poly(N-isopropylacrylamide) (carboxyl-terminated PNIPAM) by an amide group such that the carboxyl-terminated PNIPAM is linked with the molecule, an other carboxyl group of the carboxyl-terminated PNIPAM is linked with a bio-recognition molecule 2 by an amide group such that the carboxyl-terminated PNIPAM is linked with the bio-recognition molecule 2, wherein
the bio-recognition molecule 2 is a single-stranded DNA sequence 2 carrying an amino group at the 5′ end, and the single-stranded DNA sequence 2 is capable of recognizing an EpCAM protein on exosomes;
the carboxyl-terminated PNIPAM has a number average molecular weight of 1,000 to 5,000;
and luminol.

9. An application of the probe according to claim 1, the electrode of the biosensor, wherein a surface of a glassy carbon electrode (GCE) is modified by gold nanoparticles (AuNPs), the AuNPs are linked with one amino group in a molecule containing at least two amino groups by an amide group, an other amino group in the molecule is linked with one carboxyl group in a carboxyl-terminated poly(N-isopropylacrylamide) (carboxyl-terminated PNIPAM) by an amide group such that the carboxyl-terminated PNIPAM is linked with the molecule, an other carboxyl group of the carboxyl-terminated PNIPAM is linked with a bio-recognition molecule 2 by an amide group such that the carboxyl-terminated PNIPAM is linked with the bio-recognition molecule 2, wherein
the bio-recognition molecule 2 is a single-stranded DNA sequence 2 carrying an amino group at the 5′ end, and the single-stranded DNA sequence 2 is capable of recognizing an EpCAM protein on exosomes;
the carboxyl-terminated PNIPAM has a number average molecular weight of 1,000 to 5,000;
the biosensor or the kit in ECL detection of exosomes.

10. A method for detecting exosomes by ECL, the method comprising the steps of immersing the electrode of the biosensor into the exosomes solution to be detected such that the exosomes are attached to the electrode of the biosensor, then immersing the electrode of the biosensor carrying the exosomes into a solution of the probe such that the probes are attached to the exosomes on the electrode of the biosensor to constitute a biosensor consisting of the probes and the electrode of the biosensor loading the exosomes, and the modified electrode was used for subsequent ECL characterization.

11. An ECL biosensor, the biosensor comprising the probe according to claim 1, and the electrode of the biosensor, wherein the sequence of the single-stranded DNA sequence 2 from 5′ to 3′ is TTTTTT CAC TAC AGA GGT TGC GTC TGT CCC ACG TTG TCA TGG GGG GTT GGC CTG.

12. An ECL kit, the kit comprising the probe according to claim 1, and the electrode of the biosensor, wherein the sequence of the single-stranded DNA sequence 2 from 5′ to 3′ is TTTTTT CAC TAC AGA GGT TGC GTC TGT CCC ACG TTG TCA TGG GGG GTT GGC CTG, and luminol.

13. An application of the probe according to claim 1, the electrode of the biosensor, wherein the sequence of the single-stranded DNA sequence 2 from 5′ to 3′ is TTTTTT CAC TAC AGA GGT TGC GTC TGT CCC ACG TTG TCA TGG GGG GTT GGC CTG, the biosensor or the kit in ECL detection of exosomes.


SEQUENCE LISTING (2 sequences)

SEQ ID NO: 1 - DNA, 39 bp, Artificial Sequence
  synthetic single-stranded DNA sequence 1
ttttttcacccccacctcgctcccgtgacactaatgcta

SEQ ID NO: 2 - DNA, 54 bp, Artificial Sequence
  synthetic single-stranded DNA sequence 2
ttttttcactacagaggttgcgtctgtcccacgttgtcatggggggttggcctg